(19) United States
(12) Patent Application Publication
(10) Pub. No.: US 2024/0335158 A1
(43) Pub. Date: Oct. 10, 2024

(54) SOFTWARE-BASED, SPEECH-OPERATED AND OBJECTIVE DIAGNOSTIC TOOL FOR USE IN DIAGNOSING A CHRONIC NEUROLOGICAL DISORDER

(71) Applicant: VITAFLUENCE.AI GMBH, Kronberg (DE)

(72) Inventors: Peter O. OWOTOKI, Kronberg im Taunus (DE); Leah W. OWOTOKI, Kronberg im Taunus (DE); David LEHMANN, Kronberg im Taunus (DE); Diana WANJIKU, Kronberg im Taunus (DE); Moriah-Jane LORENTZ, Kronberg im Taunus (DE)

(21) Appl. No.: 18/565,820

(22) PCT Filed: May 30, 2022
(86) PCT No.: PCT/EP2022/064578

§ 371 Date: Jun. 24, 2024

(30) Foreign Application Priority Data
May 31, 2021 (DE) 10 2021 205 548.6

(51) Int. Cl. A61B 5/00; A61B 5/16

(52) U.S. Cl. CPC A61B 5/4088; A61B 5/0077; A61B 5/162; A61B 5/163; A61B 5/165; A61B 5/4803

(57) ABSTRACT

A software-based diagnostic tool and a diagnostic system for diagnosing a chronic neurological disorder. The diagnostic tool includes a speech analysis module for identifying characteristic values of a vocal biomarker in a speech signal of a subject, a further module for identifying characteristic values of a second biomarker, and an evaluation unit. The speech analysis module includes: a speech-signal-triggering which displays image data on an image display to trigger a speech signal in the subject; a speech recording unit which records the speech signal; and a speech signal analyzer that evaluates the speech signal to determine first at what point in time which pitch level is present, and subsequently determine a frequency distribution of the pitch levels among a number of frequency bands of a selected frequency spectrum, with this frequency distribution forming the characteristic values of the vocal biomarker. Based on the characteristic values of the biomarkers, the evaluation unit determines whether the subject has the chronic neurological disorder.

The invention relates to a software-based diagnostic tool for use in diagnosing a chronic neurological disorder in a human being using artificial intelligence, and to a method for operating the diagnostic tool and to a system comprising the diagnostic tool.

Chronic neurological disorders often occur in human beings. They express themselves in atypical mental development and/or atypical social behavior. Examples of such disorders are autism, attention deficit hyperactivity disorder (ADHD), schizophrenia, Alzheimer's disease, psychosis, etc. Autism is one of the best-known chronic neurological disorders, which is why it is considered below as a starting point for the invention by way of illustration but by way of representation of all chronic neurological disorders.

“Autism” is understood to mean a profound disorder in the neurological and mental development of human beings that may already occur in different severities and forms in childhood and is generally diagnosed as autism spectrum disorder, ASD for short. Autism manifests itself outwardly in particular in behavior and in communication. A striking aspect of this developmental disorder is firstly social interaction or dealings and exchanges with other human beings and also a restricted interest in repetitive, identical or similar processes, and secondly verbal and non-verbal language from the autistic person, i.e. voice and body language such as facial expressions, eye contact and gestures. A reduction in intelligence is often also discernible, but there are also forms of autism in which the person affected is averagely or even highly intelligent. This may be the case with human beings having what is known as Asperger's syndrome, which is usually accompanied by less restricted linguistic development and is therefore deemed a mild form of autism. Reports by the World Health Organization (WHO) indicate that roughly 1-2% of the world's population have an ASD, so on average 100 million people worldwide. Since this developmental disorder means that autistic people require special support and assistance in everyday life, early and correct diagnosis of this is of great importance.

Autism is conventionally diagnosed by a specialized physician, a neurologist or a therapist by asking the potentially autistic patient a varying number of specifically developed questions from a list of questions and by subsequently observing and evaluating the responses and reactions. However, it is known that only the combination of autism-specific symptoms, i.e. the symptom constellation, permits a clear diagnosis, since individual, similarly striking behavioral features also occur with other disorders.

Conventional diagnosis has several disadvantages. Firstly, it can be suggested that assessment by a medical expert is always subjective and may therefore be incorrect, specifically in both directions of diagnosis, which can have dire consequences for the patient and their relatives. This level of subjectivity, caused, among other things, by a certain prejudice, is an integral part of the evaluation process, which can lead to incorrect results in individual cases. A well-known example is the knowledge that girls are underrepresented in diagnosis because they are more adaptable and therefore show less highly pronounced behavioral problems. Another example is the preconception that autism occurs predominantly in boys, see Lockwood Estrin, G., Milner, V., Spain, D. et al., “Barriers to Autism Spectrum Disorder Diagnosis for Young Women and Girls: a Systematic Review”, Review Journal of Autism and Developmental Disorders, 2020). Even if performance of the assessment is attempted as objectively as possible, physicians or therapists require many years to acquire the necessary experience, experience that can be verbalized, taught, quantified, standardized or validated only with difficulty.

Other disadvantages are the limited availability of medical experts in terms of time and geography, the limited access to them and their diagnosis, in particular in regions of the world in which society is less widely developed, such as for example in Africa or South America, and the high costs associated with an expert diagnosis, especially since there are few experts and diagnosis is regularly performed in situ in the expert's practice, clinic or other facility of the expert. As such, affected persons and their relatives often have to accept long, arduous and costly journeys or travel to get to an expert and to be able to take advantage of their diagnosis. The global pandemic caused by the new Coronavirus SARS-CoV-2 has additionally limited the access to experts.

Regardless of this, the number of experts is low compared with demand, and so there may be long waiting times to obtain an examination appointment. Even in Germany, this waiting time may be a few years in some cases, particularly for adults, as children are given preference. In some parts of the world, e.g. in parts of Africa, children have no possibility of a diagnosis at all, on the other hand.

Finally, diagnosis on the basis of a list of questions is also disadvantageous because asking the questions takes up a lot of time, for example takes between one and three hours, and the questions and observations need to be tailored to the age, regional language and ethnic origin of the patient. The latter requires the medical expert to be familiar with the ethnic circumstances of the patient, because behavior and verbal and non-verbal communication differ from nation to nation.

The aforementioned shortcomings explained using the example of autism also apply to other chronic neurological disorders. Here too, there is a lack of sufficient experts and expert knowledge, the ability to quickly and easily reach them and above all of objective diagnosis.

The object of the present invention is to provide an apparatus, a system and a method of operation that overcome the aforementioned disadvantages and facilitate objective, at least assistive, diagnosis of a chronic neurological disorder, in particular autism and its neurological accompanying diseases, that is accessible preferably at all times and from anywhere in the world regardless of the language and ethnic origin of the affected person.

This object is achieved by a diagnostic tool having the features of claim 1, a system according to claim 18 and a method of operation according to claim 22.

Advantageous developments are specified in the respective subclaims.

The diagnostic tool according to the invention and the method applied and carried out by said diagnostic tool are based on improvements in the state of science and innovations in the field of artificial intelligence. By obtaining and evaluating specific biomarkers as objective and irrefutable proof of the presence or absence of autism, the diagnostic tool according to the invention and the method of operation for said diagnostic tool are used to produce an inexpensive, user-friendly and fast diagnosis.

A biomarker is a measurable and therefore evaluable quantity for a biological feature of a person, to be more exact a quantity that allows quantitative or quantitative assessment of a physical, physiological or behavior-typical characteristic of a person.

The invention proposes a software-based diagnostic tool for use in diagnosing a chronic neurological disorder in a human subject using artificial intelligence, comprising
a superordinate operating software,
a voice analysis module for ascertaining characteristic values of a first, specifically vocal, biomarker of a voice signal from the subject,
at least one further module for ascertaining characteristic values of a second biomarker, and
an overall result assessment unit connected downstream of the voice analysis module and the further module.

The operating software is configured to trigger the voice analysis module and the at least one further module in succession and to supply the ascertained characteristic values thereof to the overall result assessment unit.

The voice analysis module comprises
a voice signal trigger controller that is configured to present a set of individual images and/or individual videos or a text on an image display device for the subject in order to trigger at least one voice signal from the subject in the form of the naming of an object contained in the respective individual image or individual video or in the form of the reading aloud of the text,
a voice recording unit that is configured to record the voice signal in an audio recording using a voice input device, and
a voice signal analyzer that is configured to evaluate the voice signal in the audio recording initially for the purpose of determining the time at which what pitch occurs, and then to ascertain a prevalence distribution for the pitches over a number of frequency bands of a frequency spectrum considered, this prevalence distribution forming the characteristic values of the first biomarker.

The overall result assessment unit is configured to take the characteristic values of the biomarkers of the subject as a basis for applying a machine learning algorithm based on artificial intelligence to establish, through comparison with a multidimensional boundary layer, whether the subject has the chronic neurological disorder. The boundary layer may be understood to mean a mathematical hyperplane in a multidimensional space, the dimensions of which are defined by the number of characteristic values of all biomarkers. The boundary layer is a mathematical boundary between the biomarker characteristic values of persons having the chronic neurological disorder and persons without such a disorder. Looked at more closely, the overall result assessment unit is a classification model trained using biomarker characteristic values of comparison persons that establishes whether and with what level of probability the ascertained biomarker characteristic values of the subject are on the side of the boundary layer that is associated with the comparison persons having the chronic neurological disorder or on the side of the boundary layer that is associated with the comparison persons without the chronic neurological disorder.

Preferably, the learning algorithm is a support vector machine (SVM), what is known as a random forest, or a deep convolutional neural network algorithm, the learning algorithm having been trained using a number of first and second comparison datasets comprising characteristic values of the biomarkers, the first comparison datasets being associated with a group of reference persons who have the chronic neurological disorder, and the second comparison datasets being associated with a group of reference persons who do not have the chronic neurological disorder.

A special feature when using the learning algorithm is that it may be continually optimized or trained using new comparison datasets in order to perform the most accurate possible classification of the biomarker characteristic values, with the result that it becomes increasingly better at demarcating the biomarker identifiers between persons with and without a chronic neurological disorder, or at defining the boundary layer. A random forest is described in A. Paul, D. P. Mukherjee, P. Das, A. Gangopadhyay, A. R. Chintha and S. Kundu, “Improved Random Forest for Classification,” in IEEE Transactions on Image Processing, volume 27, No 8, pages 4012-4024 August 2018, for example. It is a good choice for the learning algorithm in particular if the training data, i.e. the number of comparison datasets to produce the classification model, become greater, in particular are between a few hundred and a few thousand comparison datasets. Furthermore, a deep convolutional neural network algorithm is particularly suitable if the training data, i.e. the number of comparison datasets to produce the classification model, are particularly great, in particular are above 5000, such a model even achieving a classification accuracy close to 99%.

The diagnostic tool therefore evaluates at least two biomarkers, the first biomarker (vocal biomarker) being of particular importance and denoting a characteristic of the voice of the subject. To be more exact, the first biomarker denotes the tonal spectrum used by the subject as a first criterion for judging the presence of a chronic neurological disorder. This vocal biomarker can be used to establish with 95% certainty whether the subject has a specific chronic neurological disorder. To improve the accuracy of the diagnosis, at least one second biomarker is used, the characteristic values of which are ascertained by the at least one further module.

In one variant embodiment, the further module may be an emotion analysis module for evaluating the reaction of the subject to an emotional stimulus as the second biomarker and may comprise at least the following:
an emotion trigger controller that is configured to present a set of individual images and/or individual videos or at least one single video on the image display device in order to stimulate a number of individual emotions in the subject, and
an emotion observation unit that is configured to evaluate a (video) recording of the face of the subject obtained using an image recording device at least for the purpose of determining when they show an emotional reaction.

The emotion analysis module is configured to ascertain at least the respective reaction time between the stimulation of the respective emotion and the occurrence of the emotional reaction, at least these reaction times forming the characteristic values of the second biomarker in this variant embodiment.

In another variant embodiment, the further module may be a line-of-vision analysis module for evaluating the line of vision of the subject as the second biomarker and may comprise at least the following:
a line-of-vision director that is configured to present at least one image or video on the image display device in order to direct the line of vision of the subject, and
a line-of-vision observation unit that is configured to use a (video) recording of the face of the subject obtained using an image recording device to ascertain the line of vision of said subject over time, this line-of-vision response forming the characteristic values of the second biomarker in this variant embodiment.

According to these variant embodiments, the second biomarker may therefore be a characteristic of either the emotional processing or the gaze of the subject. It therefore denotes a characteristic of their social interaction skills, specifically either the reaction time for an emotional stimulus or the line of vision, and may therefore be referred to as a “social biomarker”.

However, there is also the option of cumulatively evaluating the reaction to an emotional stimulus as a first, further biomarker and the line of vision as a second, further biomarker, with the result that the diagnostic tool examines three biomarkers altogether.

Therefore, only the voice analysis module and the emotion analysis module may be present in one variant embodiment of the diagnostic tool, only the voice analysis module and the line-of-vision analysis module in another variant embodiment, and the voice analysis module, the emotion analysis module and the line-of-vision analysis module in a third variant embodiment.

In the third variant embodiment, the emotion analysis module then forms a first further module and the line-of-vision analysis module forms a second further module, at least the reaction times for the emotional stimuli forming characteristic values of the second biomarker and the line of vision over time forming characteristic values of a third biomarker of the subject. The overall result assessment unit is then configured to take the characteristic values of the first, second and third biomarkers of the subject as a basis for applying the machine learning algorithm based on artificial intelligence to establish, through comparison with a multidimensional boundary layer (hyperplane), whether the subject has the chronic neurological disorder. The order in which the characteristic values of the second and third biomarkers are determined is not important.

Preferably, the diagnostic tool is configured to select and present the set of individual images and/or individual videos or the text for triggering the voice signal, and/or the set of individual images and/or individual videos or the at least one video for stimulating emotion and/or the at least one image or video for directing line-of-vision on the basis of person-specific data on the subject. Among other things, there may be provision for the voice signal trigger controller to be configured to take the age of the subject as a basis for selecting and presenting either the set of individual images and/or individual videos or the text. As such, children may preferably be shown the set of individual images and/or individual videos, and adults the text, on the image display device if the subject cannot read. Otherwise, the use of a text to be read aloud is preferable, because in this way the speech element is longer, sonically and tonally more comprehensive and altogether more homogeneous.

Preferably, the diagnostic tool may comprise a filter in order to filter out background or extraneous noise from the voice signal prior to pitch evaluation, in particular the voice or voices of other persons, such as for example an assistant, who might be present in the surroundings of the subject and speak during the audio recording.

Preferably, the diagnostic tool may comprise a bandpass filter that is configured to limit the pitch spectrum considered to the range between 30 and 600 Hz. Although the human voice covers a frequency range between 30 Hz and 2000 Hz, the spoken voice is usually below 600 Hz. Limiting the pitch spectrum to the range between 30 and 600 Hz for the same number of frequency bands improves the accuracy of pitch analysis because the individual frequency bands are narrower.

Preferably, the number of frequency bands is between 6 and 18, ideally is 12. This number represents good middle ground between accuracy of pitch ascertainment and processing time and processing power required therefor.

Preferably, the voice signal analyzer comprises a deep convolutional neural network algorithm in order to estimate pitches, also referred to as pitch detection in technical jargon. However, another high-quality pitch estimation algorithm may also be used, such as e.g. “PRAAT”. A crucial special feature of the voice signal analyzer, in particular the deep convolutional neural network algorithm, is its ability to learn as a result of the models for pitch estimation that it uses being continually improved and old models being able to be replaced by improved new models, whether on the basis of more available comparison data that may be used to train the models or because an intelligent optimization route has been found.

According to one variant embodiment, the emotion observation unit and/or the line-of-vision observation unit is configured to evaluate the facial recording in real time. In other words, the examination takes place while the subject is looking at the image display device or the set of individual images and/or individual videos or the at least one video or image is shown to said subject on said image display device.

Alternatively, an offline examination may take place. In this case, the emotion observation unit and/or the line-of-vision observation unit may have one video recording unit each or may use such a video recording unit that is part of the diagnostic tool to store an applicable video recording while the subject is shown the set of individual images and/or individual videos or the at least one video or image. This applicable video recording may be made available to the emotion observation unit or the line-of-vision observation unit for evaluation.

Preferably, the emotion observation unit comprises a facial recognition software based on a compassionate artificial intelligence that is trained for specific emotions, specifically, usefully, for those emotions that are stimulated by the individual images or individual videos of the set or by the video, such as e.g. joy, sadness, anger or fear.

Preferably, the emotion observation unit is configured to establish, in addition to the reaction time, the reaction type for the respective stimulated emotion, this reaction type being part of the characteristic values of the second biomarker. In the simplest case, the reaction type may be binary information indicating whether the reaction is a positive or negative emotion. By way of example, joy and sadness may be interpreted as a positive emotion, anger and fear as a negative emotion. Alternatively, the reaction type may be the specific emotion with which the subject reacts. The reaction type may then form part of the characteristic values of the second biomarker together with the applicable reaction time for the respective emotional reaction to which the reaction type is linked.

There may additionally be provision for the emotion analysis module to be configured to establish whether the reaction shown by the subject corresponds to the stimulated emotion. In the simplest case, this may be accomplished by comparing whether both the emotional stimulus and the reaction type are each a positive or each a negative emotion. If this is the case, the subject has reacted as expected, or “normally”. If this is not the case, i.e. if the emotional reaction is positive even though the emotional stimulus was negative or vice versa, the subject has reacted unexpectedly, or “abnormally”. Ideally, it is also possible to compare whether the specifically ascertained emotion with which the subject reacts corresponds to that of the stimulated emotion or these emotions are different. The result of this respective comparison may be indicated in a congruence indicator, e.g. such that a “1” indicates consistency between the emotional reaction and the stimulated emotion and a “0” indicates a lack of consistency, at least in respect of whether positive or negative emotions are involved. Alternatively, a “−1” may indicate a lack of consistency between the emotional reaction and the stimulated emotion and a “0” the fact that the subject has not shown any reaction at all. The congruence indicator may then likewise form part of the characteristic values of the second biomarker together with the applicable reaction time for the emotional reaction to which the congruence indicator is linked.

The congruence indicator is a particularly useful and informative piece of information, at any rate if the subject does not react to a specific stimulus with an emotion that would have been expected, because this is an indication of a chronic neurological disorder.

Preferably, there may be provision for the emotion analysis module to deliver three pieces of information for each stimulated emotion, specifically the reaction time for the stimulation, the emotional reaction thereto (positive/negative or specifically ascertained emotion) and the congruence indicator. These three pieces of information for each of the stimulated emotions then form the characteristic values of the second biomarker. When there are n stimulated reactions, the second biomarker comprises 3n characteristic values in this case.

Preferably, there is provision for the emotion trigger controller to be configured to stimulate between 4 and 12 emotions, preferably 6 emotions.

In one variant embodiment, the line-of-vision director may be configured to present the at least one image or video at discrete positions on the image display device in succession or to move said at least one image or video along a continuous path. The image or video is therefore displayed smaller than the display area (screen) of the image display device is, and is moved over the display area, the subject being intended to follow the sequence of display locations over time, or the display path, with their eyes. However, it is also possible to show a single video over the entire surface of the display area, in which case this video contains one or more objects whose position relative to the spatial boundary of the display area changes, e.g. a butterfly flying to and fro.

Preferably, the line-of-vision observation unit comprises an eye tracking software.

The diagnostic tool according to the invention may advantageously be used as a software application for a portable communication terminal, in particular a smartphone or tablet. The diagnostic tool can therefore be used by almost anybody at any time.

The diagnostic tool according to the invention may also be used as a software application on a server that is able to be controlled via a computer network by a browser on an external terminal in order to execute the diagnostic tool. This variant also ensures a high level of accessibility to the diagnostic tool, or access thereto at any time from any location in the world, the variant also taking account of the circumstance that the processing power in a portable communication terminal might not suffice to execute said artificial intelligence algorithms. A server having a processing unit with sufficient processing power is better suited to this.

The invention furthermore proposes a diagnostic system for use in diagnosing a chronic neurological disorder in a human subject using artificial intelligence, comprising
a diagnostic tool according to the invention,
at least one nonvolatile memory containing program code and data that form the diagnostic tool,
a processing unit such as e.g. a processor, for executing the program code and processing the data of the diagnostic tool and
the following peripheral devices:
a voice input device, such as e.g. a microphone, for recording at least one voice signal from the subject for the diagnostic tool,
an image recording device, such as e.g. a CCD camera, for graphically recording the face of the subject for the diagnostic tool,
an image display device, such as e.g. a monitor or a display, for presenting image data for the subject and
at least one input means, such as e.g. keys or a touchscreen, for the subject to make inputs,

the peripheral devices being operatively connected to the processing unit, and the diagnostic tool being configured to at least indirectly control the voice input device, the image recording device and the image display device and to evaluate the recordings from the voice input device and the image recording device.

Preferably, the diagnostic system is a portable communication terminal, in particular a smartphone or tablet, on which the diagnostic tool is executed as a software application. The nonvolatile memory, the processing unit, the voice input device, the image recording device, the image display device and the input means are integral parts of the communication terminal in this case.

Alternatively, the processing unit may be part of a server that is connected to a computer network such as the Internet and able to be controlled via a browser, the nonvolatile memory being connected to the server, and the peripheral devices being part of an external terminal, in particular a portable communication terminal. In other words, the diagnostic tool in this variant embodiment may be called via the network/Internet and executed on the server.

In a further variant embodiment, the external terminal may also have a volatile memory, the diagnostic tool being stored partly on the server memory and partly on the terminal memory. As such, by way of example, the image or text data used by the modules, and also at least the voice signal trigger controller and the voice recording unit of the voice analysis module, the emotion trigger controller of the emotion analysis module and/or the line-of-vision director of the line-of-vision analysis module, may be stored on the terminal and executed there, whereas the emotion observation unit and the reaction assessment unit and also the line-of-vision observation unit and the overall result assessment unit are stored and executed on the server memory of the voice signal analyzer. All of the computation-intensive functional units of the diagnostic tool are therefore arranged on the server. There is also the possibility of arranging all of the functional units of the diagnostic tool on the terminal except for the overall result assessment unit. This is useful firstly because the overall result assessment unit can be continually trained and therefore improved using new comparison datasets. Another advantage is that the data to be transmitted to the server, specifically the biomarker characteristic values to be assessed by the overall result assessment unit, contain no person-specific data, which means that this procedure is advantageous for data protection reasons.

The invention also proposes a method for operating the software-based diagnostic tool for use in diagnosing a chronic neurological disorder in a human subject using artificial intelligence, comprising
a superordinate operating software,
a voice analysis module for ascertaining characteristic values of a first, specifically vocal, biomarker of a voice signal from the subject,
at least one further module for ascertaining characteristic values of a second biomarker, and
an overall result assessment unit connected downstream of the voice analysis module and the further module,

wherein
the operating software triggers the voice analysis module and the at least one further module in succession and supplies the ascertained characteristic values thereof to the overall result assessment unit,
a voice signal trigger controller of the voice analysis module presents a set of individual images and/or individual videos or a text on an image display device for the subject in order to trigger at least one voice signal from the subject in the form of the naming of an object contained in the respective individual image or individual video or in the form of the reading aloud of the text,
a voice recording unit of the voice analysis module records the voice signal in an audio recording using a voice input device, and
a voice signal analyzer of the voice analysis module evaluates the voice signal in the audio recording initially for the purpose of determining the time at which what pitch occurs, and then ascertains a prevalence distribution for the pitches over a number of frequency bands of a frequency spectrum considered, this prevalence distribution forming the characteristic values of the first biomarker, and
the overall result assessment unit takes the characteristic values of the biomarkers of the subject as a basis for applying a machine learning algorithm based on artificial intelligence to establish, through comparison with a multidimensional boundary layer, whether the subject has the chronic neurological disorder.

Additionally, in one variant embodiment of the method of operation, in which the further module is an emotion analysis module for evaluating the reaction of the subject to an emotional stimulus as the second biomarker, there may be provision for
an emotion trigger controller of the emotion analysis module to present a set of individual images and/or individual videos or at least one single video on the image display device in order to stimulate a number of individual emotions in the subject, and
an emotion observation unit of the emotion analysis module to evaluate a recording of the face of the subject obtained using an image recording device (6) at least for the purpose of determining when they show an emotional reaction, and
the emotion analysis module to ascertain the respective reaction time between the stimulation of the respective emotion and its occurrence, and for at least these reaction times to form the characteristic values of the second biomarker.

As explained above, the emotion observation unit may also evaluate the recording of the face of the subject for what emotional reaction they show, i.e. the reaction type, for example to ascertain whether there is a positive or negative emotional reaction, or to determine the specific emotion. In this case, the respective reaction time and reaction type for each stimulated emotion form the characteristic values of the second biomarker.

As also explained above, the emotion analysis module may additionally ascertain a congruence indicator that indicates whether the emotional reaction corresponds to the stimulated emotion, for example whether the two are both positive or negative emotions or even the emotion type is consistent. In this case, the respective reaction line and the congruence indicator for each stimulated emotion form the characteristic values of the second biomarker. Preferably, however, the emotion analysis module ascertains three pieces of information for each stimulated emotion, specifically both the reaction time and the reaction type and congruence indicator. In this case, the respective reaction time, reaction type and congruence indicator for each stimulated emotion form the characteristic values of the second biomarker.

Additionally, in another variant embodiment of the method of operation, in which the further module is a line-of-vision analysis module for evaluating the line of vision of the subject as the second biomarker, there may be provision for
a line-of-vision director of the line-of-vision analysis module to present at least one image or video on the image display device in order to direct the line of vision of the subject, and
a line-of-vision observation unit of the line-of-vision analysis module to use a recording of the face of the subject obtained using an image recording device (6) to ascertain the line of vision of said subject over time, this line-of-vision response forming the characteristic values of the second biomarker.

Finally, in a further variant embodiment of the method of operation, there may be provision for the emotion analysis module to be a first further module and for the line-of-vision analysis module to be a second further module and for these modules to be triggered in succession, at least the reaction times for the emotional stimuli forming characteristic values of the second biomarker and the line of vision over time forming characteristic values of a third biomarker of the subject, and the overall result assessment unit taking the characteristic values of the first, second and third biomarkers of the subject as a basis for applying the machine learning algorithm based on artificial intelligence to establish, through comparison with a multidimensional boundary layer, whether the subject has the chronic neurological disorder.

Otherwise, the method of operation is configured to control the diagnostic tool such that it performs the steps and functions for which it is accordingly, as described above, configured.

The software-based diagnostic tool and the method of operation therefor are described in more detail below on the basis of a specific example and the accompanying figures, in which:

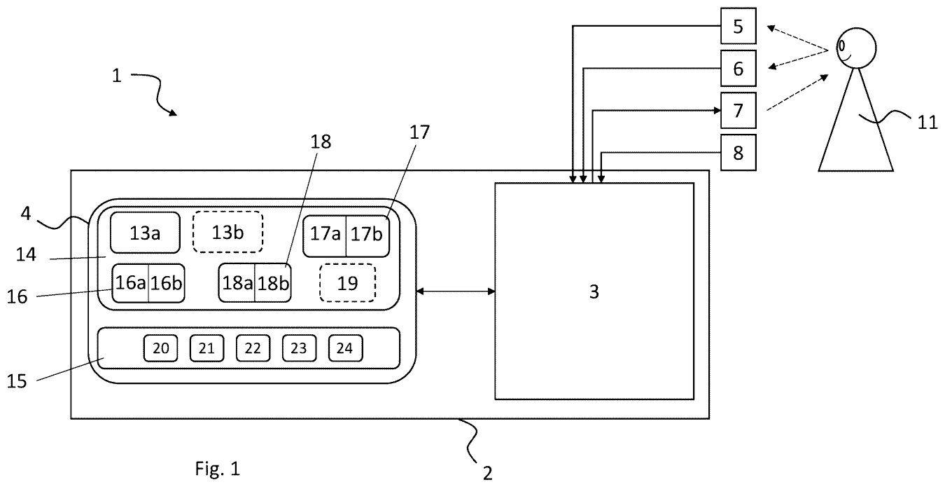

FIG. 1: shows a schematic diagram of the design of a first diagnostic system according to the invention

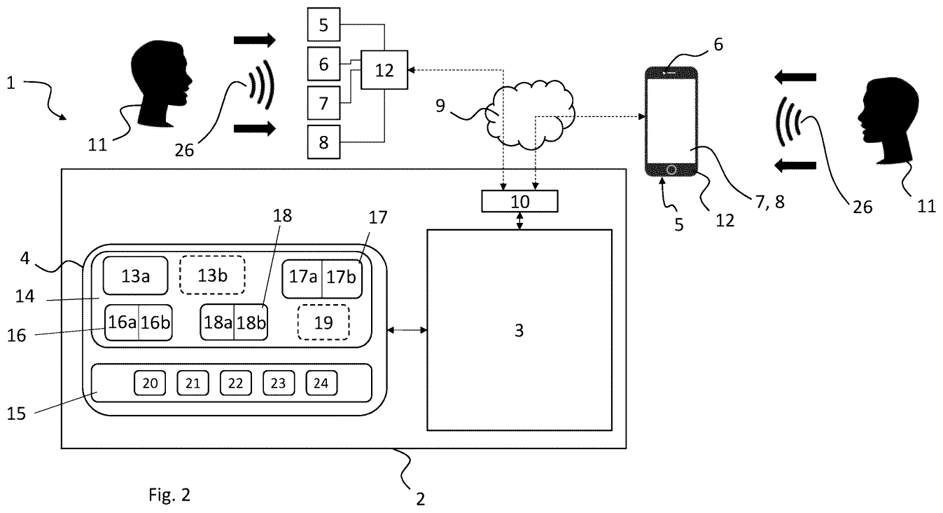

FIG. 2: shows a schematic diagram of the design of a second diagnostic system according to the invention

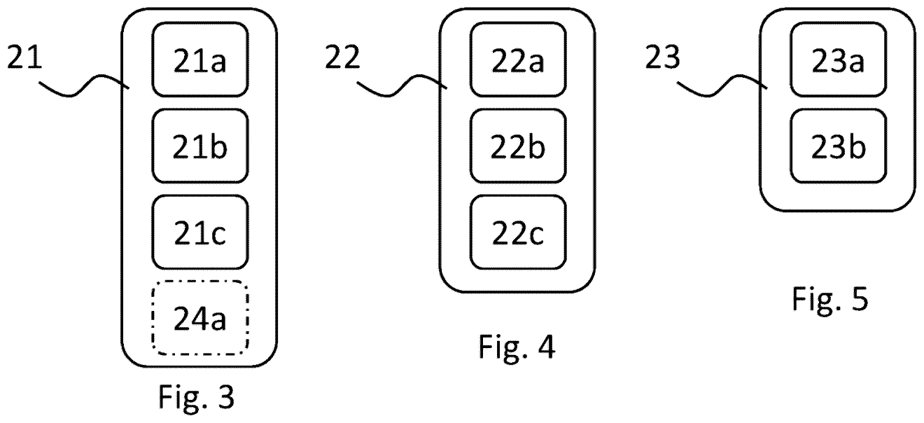

FIG. 3: shows a schematic representation of the functional units of the voice analysis module of the diagnostic tool

FIG. 4: shows a schematic representation of the functional units of the emotion analysis module of the diagnostic tool

FIG. 5: shows a schematic representation of the functional units of the line-of-vision analysis module of the diagnostic tool

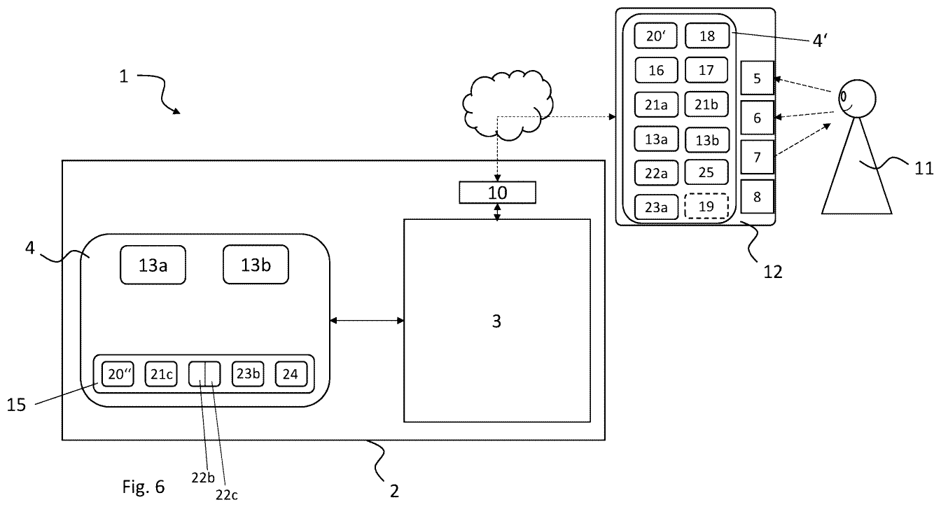

FIG. 6: shows a schematic diagram of the design of a third diagnostic system according to the invention

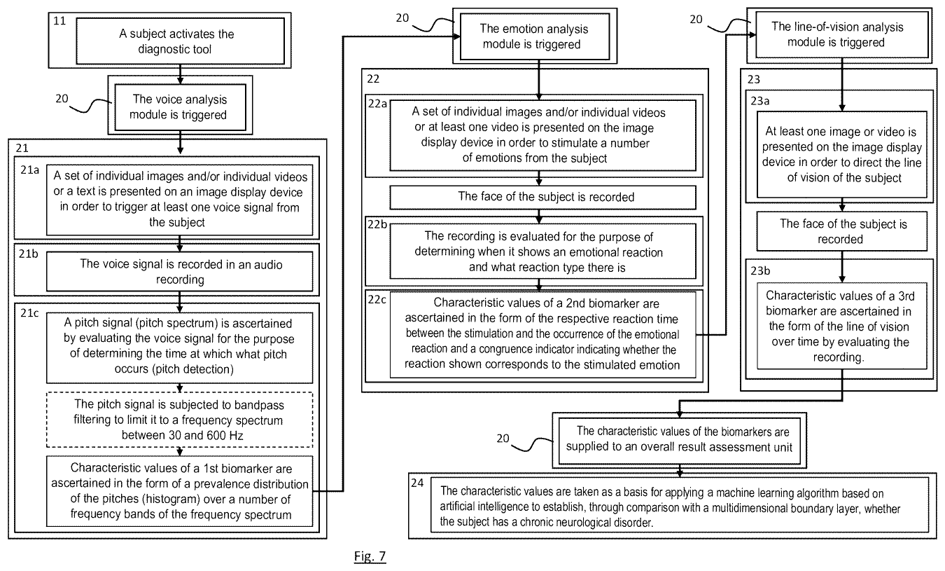

FIG. 7: shows a flowchart for a method of operation according to the invention

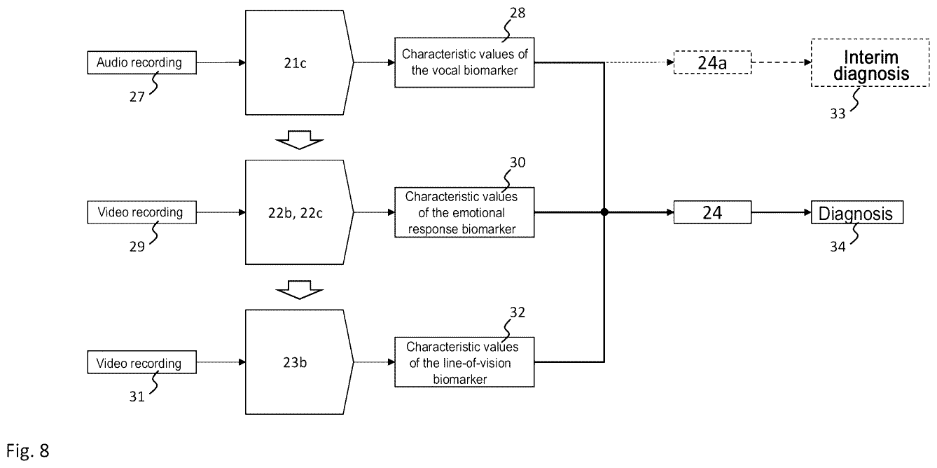

FIG. 8: shows a schematic signal flow diagram

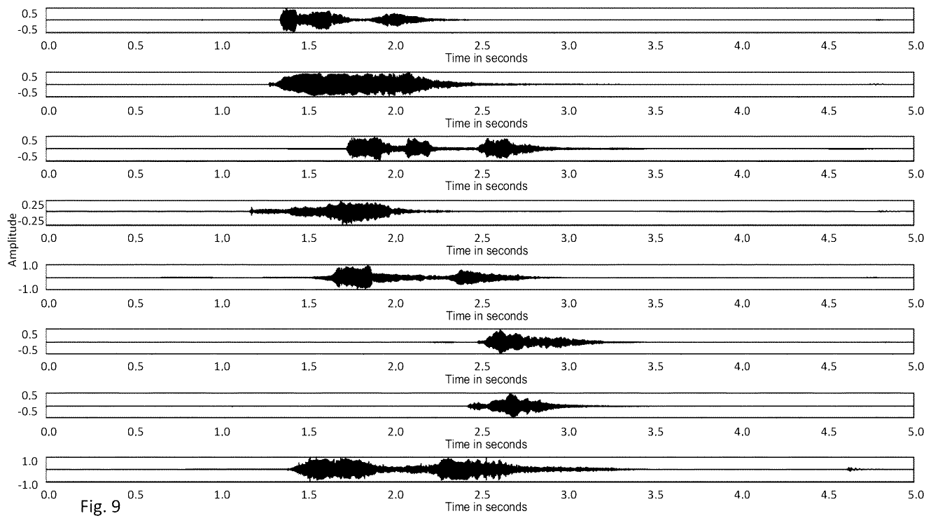

FIG. 9: shows a recorded voice signal comprising eight individual voice signals

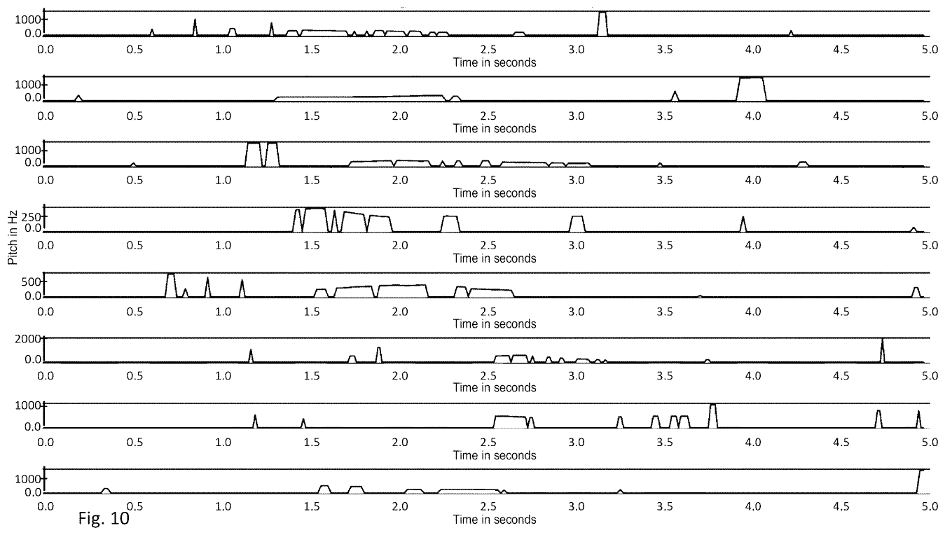

FIG. 10: shows the pitch signals of the eight individual voice signals from FIG. 9 over time (pitch spectrum)

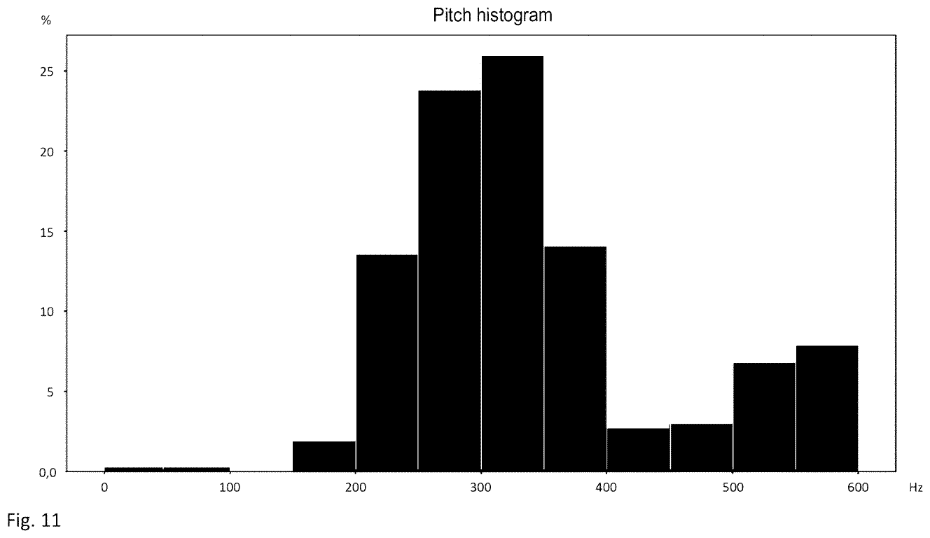

FIG. 11: shows a pitch histogram for the eight pitch signals in FIG. 10

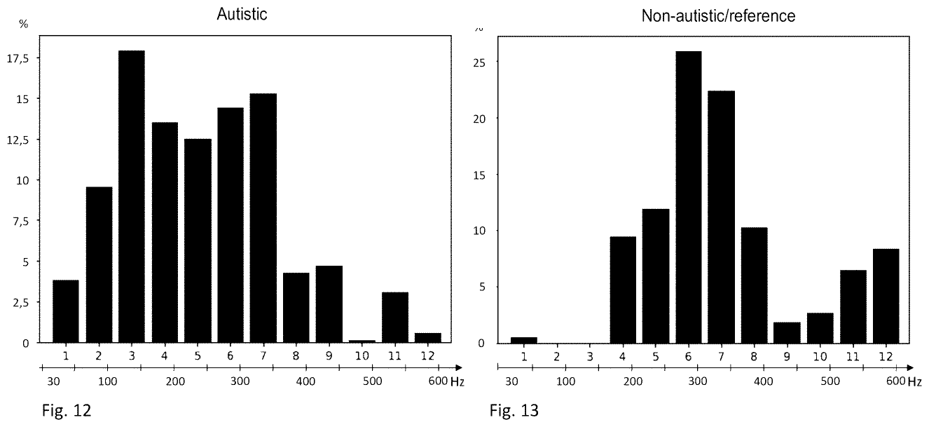

FIG. 12: shows an illustrative pitch histogram for an autistic subject

FIG. 13: shows an illustrative pitch histogram for a non-autistic subject

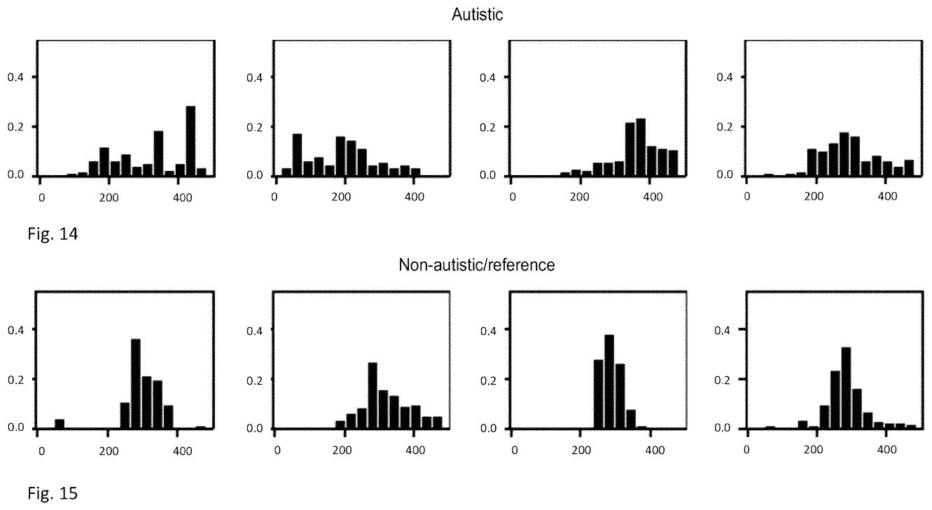

FIG. 14: shows further examples of pitch histograms for autistic subjects

FIG. 15: shows further examples of pitch histograms for non-autistic subjects

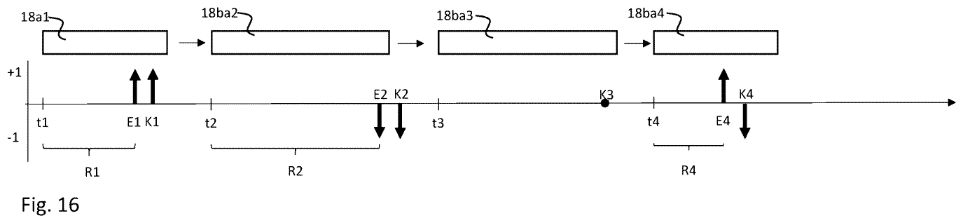

FIG. 16: shows a graph illustrating emotional stimuli and the effect thereof on the subject

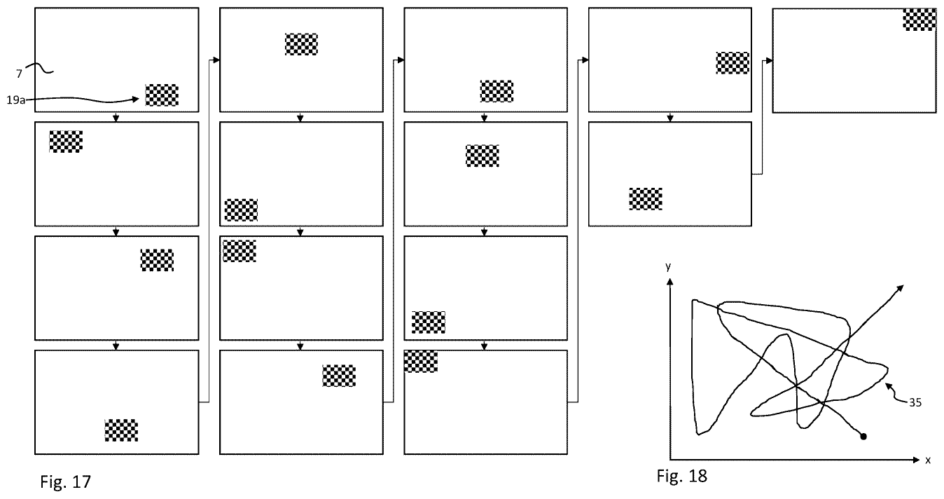

FIG. 17: shows a chronology of presentations of an image on the image display device at different positions

FIG. 18: shows an ascertained line-of-vision path

FIG. 1 shows a software-based diagnostic tool as part of a diagnostic system 1 according to a first variant embodiment. FIG. 7 illustrates a method of operation for this diagnostic tool, or for the diagnostic system. The diagnostic system 1 comprises firstly a computer system 2, which comprises at least one processing unit 3 in the form of a processor 3 having one, two or more cores, and also at least one nonvolatile memory 4, and secondly peripheral devices 5, 6, 7, 8, which are operatively connected to the computer system 2, to be more exact are connected to said computer system for communication purposes, with the result that the peripheral devices 5, 6, 7, 8 can receive control data from the computer system 2, and thus can be controlled, and/or can transmit payload data, in particular image and sound data, to said computer system.

The peripheral devices 5, 6, 7, 8 are a voice input device 5 in the form of a microphone 5, an image recording device 6 in the form of a camera 6, for example a CCD camera, an image display device 7 in the form of a display 7 or monitor, and an input means 8, e.g. in the form of control keys, a keypad or a touch-sensitive surface of the image display device 7 in conjunction with a graphical user interface presented thereon that, for a possible input, graphically highlights the subarea of the image display device 7 that is to be touched. The input means 8 may also be formed by a voice recognition module. The peripheral devices 5, 6, 7, 8 are locally associated with a subject 11, in particular are accessible to said subject, and so the subject can interact with the peripheral devices 5, 6, 7, 8.

In one variant embodiment, the peripheral devices 5, 6, 7, 8 may be connected to the computer system 2 via one or more cable connections, either via a common cable connection or via respective individual cable connections. Instead of the cable connection, the peripheral devices 5, 6, 7, 8 may also be connected to the computer system 2 via a wireless connection, in particular a radio connection such as for example Bluetooth or WLAN. A mixture of these connection types is furthermore possible, which means that one or more of the peripheral devices 5, 6, 7, 8 may be connected to the computer system 2 via a cable connection and one or more of the peripheral devices 5, 6, 7, 8 may be connected to the computer system 2 via a wireless, in particular radio, connection. In addition, the peripheral devices 5, 6, 7, 8 may be connected to the computer system directly, or indirectly via an external device 12, for example via an external computer such as a personal computer, which may in turn be connected to the computer system 2 wirelessly or by cable via at least one local and/or global network 9 such as the Internet for communication purposes. This is illustrated in FIG. 2.

The peripheral devices 5, 6, 7, 8 may each form individual devices. Alternatively, however, they may also be installed individually in combination with one another in a device. As such, for example the camera 6 and the microphone 5 may be accommodated in a common housing, or the display 7 and the input device 8 may form an integrated functional unit. As a further alternative, all of the peripheral devices may be an integral part of the external device 12, which may then be a mobile telecommunications terminal 12, for example, in particular a laptop, a smartphone or a tablet. A variant embodiment of the external device 12 in the form of a smartphone 12 is illustrated in FIG. 2. In this case too, the peripheral devices 5, 6, 7, 8 then communicate with the computer system 2 via the external device 12 and a local and/or global network 9 such as the Internet 9, to which are connected the external device 12, on one hand, wirelessly or by cable, and the computer system 2, on the other hand, wirelessly or by cable.

In the event of access from the network 9, in particular the Internet, the computer system 2 acts as a central server and, to this end, has an appropriate communication interface 10, in particular an IP-based interface, via which the communication with the external device 12 takes place. This facilitates unrestricted access to the diagnostic tool in terms of time and location. In particular, communication with the computer system 2 as the server may take place via a specific software application on the external device or via an Internet address or webpage that may be called in a browser on the external device 12.

When implementing the peripheral devices 5, 6, 7, 8 and connecting them to the computer system, there are therefore numerous physical design options allowing multiple scenarios for the use of the diagnostic tool according to the invention.

As such, the diagnostic system 1 or the computer system 2 and the peripheral devices 5, 6, 7, 8 operatively connected thereto may be situated locally at the workplace of a physician or therapist, e.g. in the practice or clinic thereof, as a common functional unit. In this case, the subject 11 needs to be present there in person in order to be to use the diagnostic system 1. It is also possible for only the external device 12 with the peripheral devices 5, 6, 7, 8, which accesses the computer system 2 or the diagnostic tool via the network 9, to be situated at said workplace. In this case, although the subject 11 still needs to be present at the physician's or therapist's in person, the investment costs for the physician or therapist are lower. However, it is particularly advantageous if the external device 12 is a mobile device, for example a laptop, smartphone or tablet, that facilitates access to the computer system 2 or to the diagnostic tool even from home. Time-consuming journeys to the physician or therapist are therefore dispensed with.

A medical expert is not necessary, in principle, in order to use the diagnostic system 1 according to the invention, since the diagnosis is performed by the diagnostic tool independently and above all objectively on the basis of the information provided to it by the subject 11 via the microphone 5 and the camera 6. The interaction between the subject 11 and the diagnostic system 1 takes place on the basis of instructions by text or voice that it outputs on the image display device 7 or a loudspeaker as a further peripheral device and with which the subject 11 needs to comply. In the case of children and such adults as are inexperienced in dealing with laptops, smartphones or tablets and the programs thereof, another person, such as a parent or a carer, may provide assistance in using the diagnostic system 1, but this does not require a medical expert. Nevertheless, the result of the diagnosis can be discussed and assessed with a medical expert, in particular in regard to any treatment resulting from a positive autism diagnosis. For reasons of emotional concern in the event of a positive autism diagnosis, it is also advisable for the diagnostic system 1 to be used under the supervision of another adult person.

In the narrower sense, the diagnostic tool according to the invention consists of a combination of software 15 and data 14 that are stored in a nonvolatile memory 4. FIGS. 1 and 2 show the simple case of the software 15 and data 14 being stored together in a memory 4 that is part of the computer system 2, for example a hard disk store. This memory 4 may also be arranged outside of the computer system 2, however, for example in the form of a network drive or a cloud. In addition, it is not imperative for the software and the data to be located in the same memory 4. Rather, the data 14 and the software 15 may also be stored in a distributed manner in different memories, inside or outside of the computer system, e.g. in a network memory or a cloud that the computer system 2 accesses as required. Furthermore, not only is it possible for the totality of the data 14 and the totality of the software 15 to be stored in separate memories, but rather parts of the data and/or parts of the software may also be stored in different memories. There are therefore numerous design options for the arrangement and distribution of the diagnostic tool within the diagnostic system 1 here too.

In particular, the data 14 of the diagnostic tool comprise image data 16, 18, 19 in the form of individual images and/or individual videos and text data 17 that are intended to be presented on the image display device 7 by the diagnostic tool in order to elicit a verbal utterance, an emotional reaction and direction of the line of vision. In view of this intended use, the image and text data 16, 17, 18, 19 are preferably each combined to form a specific group or a specific dataset, which are selected by the diagnostic tool on the basis of person-specific details of the subject.

The text data 17 are provided in order to display them on the image display device 7 to an adult who is capable of reading, as the subject 11, for the purpose of reading them aloud. The text data 17 may comprise a first text 17a in a first language, e.g. English, and a second text in a second language, e.g. Swahili. By way of example, the text may be a well known standard text, e.g. a fairytale or a story, such as e.g. Little Red Riding Hood or “A Tale of two Cities”.

A first portion 16 of the image data is provided in order to display individual images and/or individual videos on the image display device 7 in succession to an adult who is not capable of reading or to a child, as the subject 11, so that the subject 11 names the item shown in the individual images and/or individual videos. These individual images and/or individual videos 16 are designed such that only a single item that is comparatively easy to name is shown in them, such as e.g. an elephant, an automobile, an airplane, a doll, a football, etc. In the case of a video, these items may be shown moving. The individual images and/or individual videos may reflect reality or may be drawn. Since persons, in particular children, of different age, gender and ethnic origin have different interests and a different sociocultural background, individual images and/or individual videos may be divided into individual sets 16a, 16b of individual images and/or individual videos, the content of which is geared to age, gender and ethnic origin or has a specific age-related, gender-related and/or cultural context, in order to ensure that the subject 11 actually recognizes and names the respective item. The language in which naming takes place is not important, however, since it is irrelevant to the diagnostic tool.

As such, a first set of individual images 16a may be intended to be presented on the image display device 7 to a boy or a child having a first ethnic origin, and a second set of individual images 16a may be intended to be presented on the image display device 7 to a girl or a child having a second ethnic origin.

A second portion 18 of the image data is provided in order to display individual images and/or individual videos on the image display device 7 in succession to the subject 11 in order to trigger a particular emotional reaction in the subject 11, e.g. joy, sadness, anger or fear. Although individual images are suitable, in principle, for triggering an emotional reaction, such as e.g. a short comic containing a joke, videos are able to show situations that bring about more intense emotions, which is why videos are better suited in principle. In this case too, the second portion 18 of the image data 16, 18 may be divided into individual sets 18a, 18b of individual images and/or individual videos, the content of which is geared to age, gender and ethnic origin in order to ensure that the subject 11 reacts to a particular situation with a particular emotion. The individual images and/or individual videos may reflect reality or be drawn. The latter is appropriate for children.

Optionally, a third portion 19 of image data may be provided, comprising at least one individual image or video that is displayed on the image display device 7, in particular al different positions thereof in succession, to the subject 11 in order to direct their line of vision to the image display device 7. In principle, a single individual image 19a (cf. FIG. 17), which is displayed at different positions discretely in succession or is continuously moved to different positions, suffices for this purpose. In the simplest case, the individual image 19a may be any graphical object such as a symbol, an icon, a logo, a text or a figure. Alternatively, it may be a photograph or a drawing. The individual image may come from the set of individual images in the first portion 16 or second portion 18 of the image data, which means that a third portion 19 of image data is not required for directing the line of vision in this case. In order to ensure that the interest of the subject 11 in tracking the image is not lost, it is advisable to use different individual images or at least one video for directing the line of vision, which then form/s the third portion 19 of the image data. However, these individual images or the video may also come from the first portion 16 or second portion 18 of the image data, and so a third portion 19 of image data is not required in this case either.

As explained previously, the diagnostic tool comprises not only the data 14 but also software 15 (program code) containing instructions for execution on the processor 3. To be more exact, this software comprises an operating software 20, multiple analysis modules 21, 22, 23 and an overall result assessment unit 24, the operating software 20 undertaking superordinate control of the sequences in the diagnostic tool, in particular controlling the individual analysis modules 21, 22, 23 in succession and the overall result assessment unit 24, cf. FIG. 7.

The first of the analysis modules is a voice analysis module 21 for ascertaining characteristic values 27 of a first biomarker, referred to here as the vocal biomarker of a voice signal 26 from the subject 11 that is contained in an audio recording 26. In order to trigger the voice signal 26 and to obtain the audio recording 26, the voice analysis module 21 comprises a voice signal trigger controller 21a and a voice recording unit 21b. To obtain characteristic values of the vocal biomarker, a voice signal analyzer 21c is additionally part of the voice analysis module 21, as shown schematically in FIG. 3.

The voice analysis module 21, as the first analysis module, is triggered by the operating software 20 after the subject 11 or another person assisting them, such as e.g. an adult or the general practitioner, has activated the diagnostic tool, see FIG. 7, and has possibly input person-specific data, in particular relating to age, gender and ethnic origin, at the request of the diagnostic tool. There is also the possibility of these person-specific data being part of a personal profile that is already available before the diagnostic tool is started and that may be used by said diagnostic tool.

The person-specific data may be specified by the subject 11 using the input means 8. To this end, the diagnostic tool awaits an appropriate input via the input means 8 in order to subsequently select the data 14 on the basis of the input that has been made. If, however, in a simple variant of the diagnostic tool according to the invention, only a specific group of people, e.g. only adults or only children, the data may be geared specifically to this group of people and input of the person-specific data may be dispensed with. Preferably, the data or individual images and individual videos are then stored in the memory 4 following gender-neutral and ethnoculturally neutral selection.

The voice analysis module 21 is configured to first execute the voice signal trigger controller 21a. Said voice signal trigger controller is in turn configured to load a set 16a, 16b of individual images or individual videos from the first image data 16 in the memory 4, or to load a text 17a, 17b from the text data 17 in the memory 4, and to display it on the image display device 7. In the case of the individual images or individual videos, this is done in succession.

The set 16a, 16b of individual images or individual videos or the text 17a, 17b is preferably selected on the basis of the person-specific data. If the person-specific data state that the subject 11 is a child, or the age of said subject is below a specific age limit of e.g. 12 years, the set 16a, 16b of individual images or individual videos is loaded, otherwise the text 17a, 17b is loaded. This condition may also be linked to the additionally checkable condition of whether the subject 11 has reading difficulties, which may likewise be part of the person-specific data. If there are such reading difficulties, the set 16a, 16b of individual images or individual videos is likewise used. In addition, the gender and/or ethnic origin of the subject 11 may be taken as a basis for selecting a first set 16a or a second set 16b of individual images or individual videos, each of which in this respective set is tailored specifically to the applicable group of people. Furthermore, the ethnic origin or national language of the subject 11 may be taken as a basis for selecting a first text 17a or a second text 17b, each of which conforms to the applicable group of people.

The evaluation of the person-specific data, to be more exact the check as to whether the subject 11 is above the age limit, has reading difficulties, what their gender is, what ethnic origin they have or what language the subject 11 speaks or understands, and the selection of the applicable set 16a, 16b of individual images or individual videos or of the text 17a, 17b are method steps that the voice signal trigger controller 21a performs. It then loads the applicable set 16a, 16b of individual images or individual videos or the applicable text 17a, 17b from the memory 4 and controls the image display device 7 such that the individual images or individual videos in the set 16a, 16b in succession or the text 17a, 17b are/is displayed on the image display device 7.

The individual images and individual videos in the set 16a, 16b and the text 17a, 17b are intended to obtain a verbal utterance from the subject 11, subsequently called the voice signal 26. In the case of the individual images or individual videos, there is provision for the verbal utterance to be a single-word naming of the object presented in the respective individual image or in the respective individual video. In the case of the text 17a, 17b, there is provision for the verbal utterance to be the reading aloud of this text 17a, 17b. To convey this to the subject 11, there may be provision for the diagnostic tool, in particular the superordinate operating software 20 or the voice analysis module 21, to output an appropriate textual or verbal action instruction to the subject 11, for example via the image display device 7 and/or a loudspeaker, prior to display of the individual images or individual videos in the set 16a, 16b or the text 17a, 17b.

By way of example, the set 16a, 16b may comprise seven or more individual images or individual videos. The individual images or individual videos may each be displayed for a fixed period of time, e.g. for 5 s or 6 s each, with the result that, after this period has elapsed, the next individual image or individual video is displayed until all individual images or individual videos have been displayed.

At the same time as or shortly before the start of display of the individual images or individual videos in the set 16a, 16b or the text 17a, 17b on the image display device 7, the voice signal trigger controller 21a activates the voice recording unit 21b to record the voice of the subject 11 as a voice signal 26. To this end, the voice recording unit 21b switches on the voice input device 5 (microphone), records the time-continuous voice signal 26 or voice signals in an audio recording 26 and stores said audio recording in an audio data memory 13a for voice signals that are to be recorded/have been recorded. The audio recording 26 itself is digital, the voice signal 26 being able to be digitized or sampled in the voice input device 5 itself or in an analog/digital converter downstream thereof, which may be part of the processing unit 3 or of a separate digital signal processor (DSP). The audio data memory 13a may be part of the nonvolatile memory 4. It may alternatively be a memory that is separate therefrom in the computer system 2 or a memory that is separate from the computer system 2, for example a memory in a network drive or in a cloud.

The voice recording unit 21b may be configured to terminate the recording after a stipulated period in order to obtain an audio recording 27 of a certain length of time, for example 45 seconds on average for children and 60 seconds on average for adults. The voice input device 5 may then also be switched off. Alternatively, it may be switched off when the audio signal from the voice input device 5 is below a certain limit value for a specific time after a voice signal 26, i.e. the subject 11 is no longer speaking.

According to another variant embodiment, there may be provision for manual triggering and termination of the audio recording. In that case, the diagnostic tool receives an appropriate start or stop input via the input means 8.

In addition, the audio signal may ensue uninterrupted for the period of display of the individual images, individual videos or the text, and so the recording is started once, specifically at the beginning of display, and is terminated once, specifically at the end of display. It is alternatively possible to start a new audio recording for each individual image or individual video, with the result that each voice signal 26 is contained in a separate audio recording. As such, the recording may be started before or at the beginning of display of each individual image or individual video and then terminated, in particular after the voice signal 26 from the subject 11 has been obtained, either after a stipulated period of time has elapsed or if the audio signal from the voice input device 5 is below a certain limit value for a specific time after a voice signal 26. An example of individual audio recordings such as these is shown in FIG. 9.

FIG. 9 shows the responses of the amplitude, or the sound pressure level, of eight individual voice signals 26, recorded in one audio recording each, over time. The voice signals 26 are each based on an individual spoken word of greater or lesser length. The individual audio recordings may initially be processed further individually or combined to form an overall recording that is then processed further. In FIG. 8, the totality of the audio recordings is provided with the reference numeral 27, regardless of whether it is a number of individual audio recordings or a single overall recording.

The audio recording(s) 27 is/are then evaluated in the voice signal analyzer 21c, characteristic values 28 of a vocal biomarker of the recorded voice signal 26 being ascertained, cf. FIG. 8. Specifically what language and what word the subject 11 has spoken is of no significance for this, however. It is therefore not important whether the naming of the object in the respective individual image or individual video was correct.

The audio recording(s) 27 are evaluated by the voice signal analyzer 21c by first estimating the vocal fundamental frequencies or pitches in the voice signal 26 contained in the audio recording 27 using artificial intelligence over time. This is referred to as the pitch spectrum. The voice signal analyzer 21c therefore examines the basic tonal structure of the voice signal 26 in the audio recording 27. To this end, the audio recording 27 is processed in a “deep convolutional neural network” algorithm that is part of the voice signal analyzer 21c. The basic principle of such an algorithm is described in the technical paper “Luc Ardaillon, Axel Roebe: Fully-Convolutional Network for Pitch Estimation of Speech Signals” Insterspeech 2019, September 2019, Graz, Austria, 10.21437/Interspeech, 2019-2815, hal-02439798”. An example of a deep convolutional neural network algorithm for estimating the pitch spectrum is CREPE (Convolutional Representation for Pitch Estimation), which is based on a neural network having a depth of 6 convolutional layers that processes an audio signal in the time domain.

The deep convolutional neural network algorithm estimates the pitch of the audio signal 26 at each time, in particular within a specific frequency spectrum from 30 Hz to 1000 Hz that comprises all the possible tones of the human voice. The response of the pitch over time is referred to as the pitch spectrum. FIG. 10 shows the pitch spectra for the eight individual audio recordings from FIG. 9.

Experience has shown that including the frequency range above 600 Hz does not lead to any significant improvement in the analysis of the vocal biomarker, and so this frequency range can be ignored. This may be accomplished by way of bandpass filtering, for example, in which the frequency range from 30 Hz to 600 Hz is extracted from the voice signal 26. Preferably, this is done after the pitch estimation or ascertainment of the pitch spectrum, which means that the rest of the analysis is based on only the relevant portion of the human voice. This may be achieved by applying a digital bandpass filter, which is likewise part of the voice signal analyzer 21c, to the audio recording(s) 27. In one variant embodiment, this bandpass filter may have fixed cut-off frequencies, in particular at 30 Hz and 600 Hz. Alternatively, the bandpass filter may have variable cut-off frequencies, there being provision for the minimum and maximum frequencies in the pitch spectrum to be determined and then for the bandpass filter to be configured such that the lower cut-off frequency corresponds to the ascertained minimum frequency and the upper cut-off frequency corresponds to the ascertained maximum frequency.

Before the audio recording is processed in the deep convolutional neural network algorithm, the voice signal 26 may additionally be filtered such that background noise in the voice signal 26, such as e.g. the voice of persons other than the subject 11, is eliminated. This may be achieved by applying an appropriate digital filter, which is likewise part of the voice signal analyzer 21c, to the audio recording(s) 27. Digital filters of this type are known per se. Background noise is usefully filtered out before the pitch estimation or ascertainment of the pitch spectrum so that the result of this estimation is not distorted.

A histogram analysis is subsequently applied to the pitch spectrum of the audio recording(s). A histogram that is the result of this analysis is shown in FIG. 11. The histogram analysis involves the frequency range under consideration, here the range between 30 Hz and 600 Hz, being divided into a number n of identical segments that each form a container. Each individual pitch ascertained in the audio recording over time is then assigned to the applicable segment or container. This is consistent with range-related summation of the occurrences of the individual pitches. In other words, each frequency segment is taken in order to ascertain how often one of its pitches is contained in the audio recording. The ascertained number of respectively summed pitches in each segment is then divided by the number of pitches ascertained in total. The histogram therefore provides a % indication of how often the pitches or frequencies of a specific frequency segment occurred in the audio recording. When there are multiple individual audio recordings, as shown in FIG. 9, this is accomplished by looking at the totality of all the audio recordings or the totality of all the pitch spectra (FIG. 10). In the present example, the relevant frequency range has been divided into 12 segments, it also being possible for there to be more or fewer.

Example: if a pitch of 320 Hz occurs in the audio recording 27, this is assigned to the 7th segment. If a further pitch occurs at 280 Hz, this is assigned to the 6th segment. A pitch of 340 Hz is again assigned to the 7th segment, etc. If the same pitch, e.g. 320 Hz, occurs another time, it is again assigned to the 7th segment. If these four pitches were to be all, the 6th segment would have one assignment and the 7th segment would have three assignments, resulting in a prevalence of 25% for the frequency range between 250 Hz and 300 Hz (6th segment) and a prevalence of 75% for the frequency range between 300 Hz and 350 Hz (7th segment). A histogram illustrates these prevalences.

In the pitch histogram in FIG. 11, for example the frequency range between 200 Hz and 250 Hz (5th segment) is represented with a prevalence of approximately 13%, the frequency range between 250 Hz and 300 Hz (6th segment) is represented with a prevalence of approximately 23.5%, the frequency range between 300 Hz and 350 Hz (7th segment) is represented with a prevalence of approximately 26% and the frequency range between 350 Hz and 400 Hz (8th segment) is represented with a prevalence of approximately 14%.

FIGS. 12 and 13 each show a further pitch histogram as the result of a histogram analysis. The histogram in FIG. 12 belongs to a voice signal from a demonstrably autistic subject 11, whereas the histogram in FIG. 13 belongs to a voice signal from a demonstrably non-autistic reference person. The histogram provides a statement about the pitch variability in the voice of the subject 11, this being an objective biomarker to distinguish an autistic person 11 from a reference person without autism. As FIGS. 12 and 13 illustrate when compared with one another, the pitch of the voice varies to a lesser extent in a non-autistic person; it is limited more to specific frequencies. The frequencies used are in a comparatively narrow frequency band here, specifically between 250 Hz and 400 Hz, and have a clear peak there, specifically at approximately 300 Hz, see FIG. 13. By contrast, the variability of the pitch of the voice is greater for an autistic person, as FIG. 11 shows. Here, the dominant frequencies extend over a distinctly wider frequency band, specifically between 50 Hz and 350 Hz, see FIG. 12, and the distribution thereof is more uniform, i.e. it has no clearly pronounced peak.

This knowledge is also verified by the histograms of autistic subjects in FIG. 14 compared with the histograms of non-autistic reference persons in FIG. 15. FIGS. 14 and 15 each show four histograms. It can clearly be seen that autistic people use a broader tonal spectrum.

The pitch histogram may be understood as a vocal biomarker. The characteristic values of this biomarker are formed by the frequencies of occurrence of the n frequency segments in this case. In other words, the histogram analysis shown in FIG. 11 delivers twelve characteristic values, i.e. one frequency of occurrence for each frequency segment.

The histogram or the characteristic values of this biomarker may then be evaluated in a preliminary assessment unit 24a to determine whether the subject 11 is non-autistic, cf. FIG. 8. This can be established with more than 95% certainty. However, the vocal biomarker alone is not informative enough to be able to make an unequivocally positive diagnosis of autism, and so further examinations are required, as explained below. The result of the preliminary assessment unit 24a is therefore the interim diagnosis 33 that the subject 11 is unequivocally non-autistic, or requires further examination.

The preliminary assessment unit 24a may likewise be part of the voice analysis module 21, cf. FIG. 3, or else formed by the overall assessment unit 24 used for the interim diagnosis 33. It will be noted that the interim diagnosis 33 by the preliminary assessment unit 24a is not absolutely necessary. Rather, there may be provision for each subject 11 to perform all the analyses offered by the diagnostic tool.

The preliminary assessment unit 24a is an algorithm that compares the characteristic values with a multidimensional plane, also called a hyperplane, which, in graphical terms, forms a boundary layer between subjects with and subjects without autism in a multidimensional data space. The algorithm may be a machine learning algorithm or preferably a support vector machine (SVM). Such algorithms are known generally, for example from Boser, Bernhard E.; Guyon, Isabelle M.; Vapnik, Vladimir N. (1992). “A training algorithm for optimal margin classifiers”. Proceedings of the fifth annual workshop on Computational learning theory—COLT '92. p. 144, or Fradkin, Dmitriy; Muchnik, Ilya (2006). “Support Vector Machines for Classification”. In Abello, J.; Carmode, G. (eds.). Discrete Methods in Epidemiology. DIMACS Series in Discrete Mathematics and Theoretical Computer Science. 70. pp. 13-20. These involve a model that has been trained using datasets of vocal biomarkers of a multiplicity of reference persons with and without autism, meaning that the model is able to associate the ascertained characteristic values 28 of the vocal biomarker of the subject 11 with a person with autism or with a person without autism with a high degree of accuracy, the association accuracy for subjects 11 without autism being more than 95%.

If the result of the interim diagnosis 33 is that the subject 11 is not unequivocally non-autistic, or if an interim diagnosis 33 is dispensed with, the analysis of the vocal biomarker is followed by the analysis of a further biomarker, either in the form of the reaction time of the subject 11 to an emotional stimulus, or in the form of the line of vision of the subject 11, both of said further biomarkers preferably being analyzed, and a particular order not being important.

According to one variant embodiment, the operating software 20 activates the emotion analysis module 22 after the voice analysis module 21, see FIG. 7. This may be done automatically or on the basis of a corresponding input from the subject 11 that the diagnostic tool is expecting. The emotion analysis module 22 comprises an emotion trigger controller 22a, an emotion observation unit 22b and a reaction assessment unit 22c, cf. FIG. 4. The emotion analysis module 22 measures the reaction time of the subject 11 to an emotional stimulus that is triggered for the subject 11 by the presentation of selected image data 18 on the image display device 7 in the form of individual images or individual videos, the measurement being performed by applying a facial recognition software and compassionate artificial intelligence (compassionate AI) that is capable of recognizing certain emotions in a face. Preferably, this artificial intelligence is what is known as a “deep learning model” that has been trained using representative datasets for the emotions that are to be stimulated.

The emotion analysis module 22 does this by starting the emotion trigger controller 22a in a first step. Said emotion trigger controller is configured to load a set 18a, 18b of image data 18 from the memory 4 and to display said set, or to have it displayed, on the image display device 7. As in the case of the voice analysis module 21, a set 18a, 18b may be selected from multiple sets on the basis of the aforementioned person-specific data, and so children, or girls or persons with a first ethnic origin are shown a first set 18a of the image data 18, and adults, or boys or persons with a second ethnic origin are shown a second set 18b of the image data 18. These image data 18 are a number of individual images or individual videos shown on the image display device 7 in succession. Their content is chosen such that it triggers an emotional reaction in the subject in the form of joy, cheerfulness, sadness, fear or anger.

Fittingly, the image dataset 18 comprises a total of 6 individual images and/or individual videos that each stimulate the same number of positive emotions such as joy or cheerfulness and negative emotions such as sadness, fear or anger.

At the same time as or shortly before display of the first individual image or video, the emotion trigger controller 22a activates the emotion observation unit 22b, which in turn activates the image recording device 7 to capture, and if necessary, at least temporarily, also record, the face of the subject 11 or their facial expression. In one variant embodiment, the emotion observation unit 22b may be configured to record the captured face in a video recording and to analyze it “offline”, i.e. after the whole set 18a, 18b of individual images or videos has been shown. Alternatively, a realtime evaluation of the face captured by the image recording device 7 may be performed, and so a video recording does not need to be stored. FIG. 8 shows a video recording 29 that represents the output signal from the image recording device 7 and may be either a stored video recording or a realtime recording, which is supplied to the emotion observation unit 22b by way of signaling.

With every display of a new individual image or individual video, the emotion trigger controller 22a may set a start timestamp t1, t2, t3, t4 that is later used as a reference. FIG. 16 illustrates this on the basis of four individual videos 18a1, 18a2, 18a3, 18a4 from the first set 18a of the image data 18, which are shown in succession. Said facial recognition software with compassionate artificial intelligence is part of the emotion observation unit 22b, which evaluates the video recording 29 for the purpose of determining when the facial features of the subject 11 change beyond a measure that can be unequivocally associated with an emotional reaction, in particular a specific expected emotion. In each of these recognized cases, the emotion observation unit 22b sets a reaction timestamp E1, E2, E4. From the difference between the respective reaction timestamp E1, E2, E3, E4 and the corresponding previously set start timestamp t1, 12, 13, 14 as a reference, the reaction time R1, R2, 84 (Ri=Ei−ti, where i=1, 2, 4) is then ascertained for each of the stimulated emotions. This takes place in the reaction assessment unit 22c. In the example shown in FIG. 16, it has been assumed that the subject 11 shows no or insufficient emotion for the third individual video 18a3, which means that no reaction timestamp has been able to be set here either.

The single individual images or individual videos 18a1, 18a2, 18a3, 18a4 may be displayed by the emotion trigger controller 22a for a specific stipulated period, the individual periods being able to be identical or different. The next individual image or video is therefore shown when the period for the previous image has elapsed. Alternatively or additionally, the next individual image or individual video may be shown as soon as or shortly after the emotion observation unit 22b has recognized an emotion. In this case, the emotion observation unit 22b provides feedback to the emotion trigger controller 22a to show the next individual image or individual video. Since, however, there is the risk of a specific emotion not being triggered in the subject 11 at all, termination of the display of an individual image or individual video after the relevant period forms a necessary fallback position. The non-stimulation of an emotion is likewise recorded in said individual image or individual video by the reaction assessment unit 22c, e.g. with the value zero.

In one variant embodiment, there may be provision for the emotion trigger controller 22a to trigger a timer instead of the start timestamps, the emotion observation unit 22b being able to stop the timer again on recognizing an emotion, instead of setting the reaction timestamps. The timer value is then read by the reaction assessment unit 22c and stored, since it represents the respective reaction time for the specific stimulated emotion. If a particular emotion is not triggered in the subject 11, the timer may be reset when the next individual image or individual video is displayed or at the end of the display period for the last individual image or individual video. This instance of non-stimulation of an emotion is also recorded by the reaction assessment unit 22c.

In addition to determining an emotional reaction, the emotion observation unit 22b may also be configured to establish whether a positive or negative emotion has been stimulated in the subject 11. This finding, called the reaction type below, may be represented in the form of binary information +1 or −1 and linked to the applicable reaction time R1, R2, R4. It is used for plausibility checking, or allows the congruence of the emotional reaction with the stimulated emotion to be determined.

There may therefore additionally be provision for the reaction type to be used to establish whether the subject 11 shows a reaction that can be expected for the stimulation. This is Illustrated in FIG. 16 on the basis of a congruence indicator K1, K2, K3, K4. Said congruence indicator is obtained from the result of a comparison to determine whether the determined reaction type corresponds to the stimulated emotion. This comparison may likewise be performed by the reaction assessment unit 22c. If the reaction type and the stimulated emotion are each positive or each negative, there is congruence or consistency. This case may be indicated by the congruence indicator using the value 1. Referring to FIG. 16, the subject 11 reacts to the emotions stimulated by the first two individual images or individual videos 18a1, 18a2 as expected, which means that the first and second congruence indicators K1, K2 each have the value 1. If the reaction type and the stimulated emotion are different, there is a lack of congruence or consistency. This case may be indicated by the congruence indicator using the value 0 or −1. In FIG. 16, the value −1 is chosen so that the congruence indicator value 0 may be used to indicate that there has been no reaction. This is the case with the third individual image or individual video 18a3, for which the third congruence indicator K3=0. In the example in FIG. 16, the subject 11 reacts to the emotion stimulated by the fourth individual image or individual video 18a4 unexpectedly. Here, the type of emotional reaction is not consistent with the stimulated emotion, and so the fourth congruence indicator K4 has the value −1.

As an alternative to the variant described above with individual images and/or individual videos, there may be provision for one single video containing the individual emotional stimuli at specific times that are known to the emotion trigger controller 22a to be shown on the image display device 7 by the emotion trigger controller 22a. It is therefore not necessary for starting timestamps to be set.

For each of a number of emotions stimulated in the subject 11, the emotion analysis module 22 therefore delivers one reaction time Ri, one reaction type (positive or negative emotion, +1, −1) and one congruence indicator (values −1, 0, +1), which, in their totality, form the characteristic values 30 of the second biomarker, called the “emotion response biomarker” in FIG. 8. A table of these characteristic values is shown below:

| | Stimulus 1 | Stimulus 2 | Stimulus 3 | Stimulus 4 |
|---|---|---|---|---|
| Reaction type | +1 | −1 | — | +1 |
| Reaction time | R1 | R2 | ∞ | R4 |
| Congruence | +1 | −1 | 0 | −1 |
| indicator | | | | |

After the characteristic values 30 of the second biomarker have been ascertained by the emotion analysis module 22, the operating software 20 activates the line-of-vision analysis module 23, which is responsible for ascertaining characteristic values 32 of a third biomarker of the subject 11, see FIG. 7. This may be done automatically or on the basis of a corresponding input from the subject 11 that the diagnostic tool is expecting. The line-of-vision analysis module 23 comprises a line-of-vision director 23a and a line-of-vision observation unit 23b, cf. FIG. 5. The line-of-vision analysis module 23 measures and tracks the line of vision of the subject 11 while they are looking at the image display device 7. The image recording device 6 is usefully arranged relative to the image display device 7 such that it captures the face of the subject 11.

The line-of-vision analysis module 23 does this by starting the line-of-vision director 23a in a first step. Said line-of-vision director is configured to load at least one individual image 19a or video in the image data 16, 18, 19 from the memory 4 and to display said image or video, or to have it displayed, on the image display device 7. As in the case of the voice analysis module 21 and the emotion analysis module 22, the at least one individual image 19a or video may be selected on the basis of the aforementioned person-specific data.

Different variant embodiments are conceivable for directing the gaze of the subject 11. In the case of an individual image 19a, said individual image is displayed on the image display device 7 in a smaller size than its total area or resolution allows, which means that the individual image 19a fills only a portion of the display screen of the image display device 7. It is then shown in this form by the line-of-vision director 23a at different positions on the display screen at successive times, the individual image 19a being able to appear discretely at these positions in succession or to be moved continuously from position to position along a continuous path. This variant is Illustrated in FIG. 17.

In the case of discrete appearance, instead of a single individual image it is also possible for two or more different individual images in the image data 16, 18, 19 to be loaded from the memory 4 and displayed alternately or at random at different screen positions on the image display device 7. Furthermore, it is possible to use one or more videos instead of the individual image(s), said video(s) being displayed at the individual positions in succession. Therefore, the display size of this video or these videos is also smaller than the total area or resolution of the image display device 7.

The subject 11 is required to attentively follow the display location of the individual image or the individual images. To this end, the diagnostic tool may output an appropriate prompt on the image display device 7 or via a loudspeaker beforehand.

Instead of the one or more individual images, the line-of-vision director 23a may show a video on the image display device 7, specifically over the entire surface, that is designed to direct the gaze of the subject 11 along a specific path over the image display device 7. To this end, the video may contain an object moving relative to a stationary background, for example, such as e.g. a clownfish moving in an aquarium. Alternatively, events that draw the attention of the subject may occur in the video at spatially different locations at successive times. In these cases, the line-of-vision director 23a therefore requires just this one video.

At the same time as or shortly before the individual image or video is displayed, the line-of-vision director 23a activates the line-of-vision observation unit 23b, which in turn activates the image recording device 7 to capture, and if necessary, at least temporarily, also record, the face of the subject 11 or their line of vision. In one variant embodiment, the line-of-vision analysis module 23 may be configured to record the captured face in a video recording and to analyze it “offline”, i.e. after the at least one individual image or video has been shown. Preferably, however, the line of vision of the face captured by the image recording device 7 is evaluated in real time, and so a video recording does not need to be permanently stored. FIG. 8 shows a video recording 31 that represents the output signal from the image recording device 7 and may be either a stored video recording or a realtime recording, which is supplied to the line-of-vision observation unit 23b by way of signaling.

The line-of-vision observation unit 23b is formed by an eye tracking software based on artificial intelligence. Such a software is well known, for example from Krafka K, Khosla A, Kellnhofer P, Kannan H., “Eye Tracking for Everyone”, IEEE Conference on Computer Vision and Pattern Recognition. 2016; 2176-2184. It ascertains the line of vision of the subject 11 in the form of x, y coordinates of the eye focus at any time and stores it, with the result that a line-of-vision path 35 is obtained over time, as shown in FIG. 18. Said line-of-vision path represents the characteristic values 32 of the third biomarker, called the line-of-vision biomarker below, cf. also FIG. 8. The coordinates may be those of the real space or referenced to the display area of the image display device 7.

As FIG. 8 shows, the characteristic values 28, 30, 21 of the three biomarkers, to be more exact the vocal biomarker, the emotional response biomarker and the line-of-vision biomarker, are supplied to an overall result assessment unit 24 that is part of the diagnostic tool according to the invention and that combines the characteristic values 28, 30, 21 of the biomarkers. Like the preliminary assessment unit 24a, the overall result assessment unit 24 is an algorithm based on artificial intelligence and in the form of a model that has been trained using datasets of the three biomarkers of a large number of reference persons with and without autism. Looked at closely, the algorithm is a classification algorithm that classifies the biomarkers of the subject as “autistic” or “non-autistic” with a certain level of probability. The algorithm may be a machine learning algorithm or preferably a support vector machine (SVM). It compares the totality of all the characteristic values 28, 30, 21 of the three biomarkers at the same time with a hyperplane that forms a boundary layer between subjects with and subjects without autism in a multidimensional data space, in order to obtain an association between the totality of the data formed by the characteristic values and either a reference group comprising persons with autism or a reference group comprising persons without autism. On the basis of this association result, the diagnosis 34 obtained is then that the subject 11 has a certain probability of being autistic or non-autistic.

The assistive use of the diagnostic tool according to the invention in diagnosing autism allows more than 95% accuracy in establishing whether a subject 11 suffers from autism. The evaluation of the biomarkers leads to a robust and above all objective result. Among the large number of people who potentially suffer from autism and are waiting for a diagnosis by a medical expert, use of the diagnostic tool allows a contribution to be made to reducing the diagnosis backlog and allows the decision regarding which patients should be given preference for diagnosis by the medical expert to be simplified.

A particular advantage of the diagnostic tool is that both adults and children may be examined using it and use of the diagnostic tool is possible from almost anywhere and at any time, in particular from home.

As explained with reference to FIG. 1, the software-based diagnostic tool is part of a diagnostic system 1. In a first variant embodiment, said diagnostic system may be a computer system 2 having peripheral devices connected thereto, in particular a microphone 5, a camera 6, a display/monitor and an input means 8. The computer system 2 itself may be a personal computer having a nonvolatile memory 4 in which the diagnostic tool consisting of the aforementioned software components or modules and data is stored.

In a second variant embodiment, which is shown in FIG. 2, the computer system 2 may act as a server that can be reached via the Internet 9 using an external, in particular mobile, device 12. In this case, the peripheral devices 5, 6, 7, 8 are part of the external device, which is a smartphone or a tablet, for example. In this case, as previously, the diagnostic tool is formed by the software components or modules and data stored in the memory 4 of the computer system 2.

In a third variant embodiment, which is illustrated in FIG. 6, the diagnostic tool may be in a distributed arrangement, to be more exact may be formed partly in the computer system 2 and partly in the external device 12. This variant embodiment produces an offline analysis of the biomarkers. As such, the external device 12 may contain a nonvolatile memory 4′ and a processor, which is not shown here. The nonvolatile memory 4′ stores the image data 16, 18, 19 and the text data 17, on the one hand, and also a part 20′ of the operating software and those components 21a. 21b, 22a, 23a of the analysis modules 21, 22, 23 that do not have high processing power and make no special demands on the processor, e.g. a multicore processor. The memory 4′ stores the voice signal trigger controller 21a and the voice recording unit 21b of the voice analysis module 21. They carry out the same method as explained previously, but one difference is that the audio recording 27 is stored in the audio data memory 13a and not analyzed on the external device 12. Furthermore, the memory 4′ stores the emotion trigger controller 22a of the emotion analysis module 22 and the line-of-vision director 23a of the line-of-vision analysis module 23. These each also carry out the same method as explained previously, one difference being that during the respective display of the image(s) a video recording 29, 31 is made that is stored in the video data memory 13b and not analyzed on the external device 12. To record the video recordings 29, 31, the memory 4′ additionally contains, analogously to the voice recording unit 21b, a video recording unit 25.

By contrast, the memory 4 of the computer system 2 contains not only a second part 20′ of the operating software but also just those components of the analysis modules 21, 22, 23 that perform the actual analysis of the biomarkers, specifically the voice signal analyzer 21c of the voice analysis module 21, the emotion observation unit 22b and the reaction assessment unit 22c of the emotion analysis module 22 and the line-of-vision observation unit 23b of the line-of-vision analysis module 23. Finally, however, the overall result assessment unit is also present in the memory 4. In addition, there is also provision in the memory 4 of the computer system 2 for an audio data memory 13a and a video data memory 13b, to which the audio and video recordings 27, 29, 31 stored on the external device 12 are transmitted. This may take place in each case immediately after storage of the applicable recording or else only after all the recordings have been made. The evaluation of the individual biomarkers and the collective assessment of their characteristic values then continue to take place on the computer system.

In a fourth variant embodiment, which is not shown, there may be provision, as a development of the third variant, for the analyzing components 21c, 22b, 22c, 23b of the analysis modules 21, 22, 23 to also be arranged in the external device 12, which means that the characteristic values 28, 30, 32 of the biomarkers are likewise ascertained in the external device 12. As a result, only these characteristic values 28, 30, 31 are then transmitted to the computer system 2, where they are collectively evaluated as appropriate using the overall result assessment unit 24. This is advantageous for data protection reasons, since the characteristic values of the biomarkers do not allow the subject to be identified.

In a fifth variant embodiment, there is provision for the diagnostic tool to be arranged entirely in the external device 12, which means that the diagnostic system 1 is formed only from this external device 12 with the peripheral devices 5, 6, 7, 8 already integrated therein and the diagnostic tool stored thereon. The diagnostic tool may be implemented in an application, called an app for short, and executed on an applicable processor of the external device. The external device is preferably a smartphone or tablet.

### LIST OF REFERENCE SIGNS

1 diagnostic system
2 computer system/server
3 processing unit/processor
4, 4′ nonvolatile memory
5 voice input device/microphone
6 image recording device/camera
7 image display device/display
8 input means/control keys
9 network/Internet
10 interface
11 subject
12 external, optionally mobile, terminal, tablet/smartphone/laptop
13a audio data memory for voice elements that are to be stored/have been stored
13b video data memory for video recordings that are to be recorded/have been recorded
14 data
15 program code/software
16 first image data, for triggering voice
16a first set of individual images or videos to be displayed
16b second set of individual images or videos to be displayed
17 text data of texts to be displayed
17a first set of text data
17b second set of text data
18 second image data, for triggering emotion
18a first set of individual images or videos to be displayed
18b second set of individual images or videos to be displayed
18a1, 18a2, 18a3, 18a4 individual videos
19 third image data, for directing line of vision
19a individual image
20, 20′, 20″ operating software
21 voice analysis module
21a voice signal trigger controller
21b voice recording unit
21c voice signal analyzer
22 emotion analysis module
22a emotion trigger controller
22b emotion observation unit
22c reaction assessment unit
23 line-of-vision analysis module
23a line-of-vision director
23b line-of-vision observation unit/eye tracking
24 overall result assessment unit
24a preliminary assessment unit
25 video recording unit
26 voice signal
27 audio recording
28 characteristic values of the vocal biomarker
29 first video recording
30 characteristic values of the emotional response biomarker
31 second video recording
32 characteristic values of the line-of-vision biomarker
33 assessment result/interim diagnosis
34 assessment result/diagnosis
35 line-of-vision path

1-25. (canceled)

26. A software-based diagnostic tool for use in diagnosing a chronic neurological disorder in a human subject using artificial intelligence, comprising:
a superordinate operating software;
a voice analysis module for ascertaining characteristic values of a first, specifically vocal, biomarker of a voice signal from the subject;
at least one further module for ascertaining characteristic values of a second biomarker; and
an overall result assessment unit connected downstream of the voice analysis module and the further module,
wherein the operating software is configured to trigger the voice analysis module and the at least one further module in succession and to supply the ascertained characteristic values to the overall result assessment unit,
wherein the voice analysis module comprises:
a voice signal trigger controller that is configured to present a set of individual images and/or individual videos or a text on an image display device for the subject to trigger at least one voice signal from the subject in the form of the naming of an object contained in a respective individual image or individual video or as a reading aloud of the text;
a voice recording unit configured to record the voice signal in an audio recording using a voice input device, and
a voice signal analyzer configured to evaluate the voice signal in the audio recording initially for determining a time at which what pitch occurs, and then to ascertain a prevalence distribution for the pitches over a number of frequency bands of a frequency spectrum considered, the prevalence distribution forming the characteristic values of the first biomarker, and
wherein the overall result assessment unit is configured to take the characteristic values of the biomarkers of the subject as a basis for applying a machine learning algorithm based on artificial intelligence to establish, through comparison with a multidimensional boundary layer, whether the subject has the chronic neurological disorder.

27. The diagnostic tool according to claim 26, wherein the at least one further module is an emotion analysis module that evaluates a reaction of the subject to an emotional stimulus as the second biomarker, wherein the emotion analysis module comprises:
an emotion trigger controller configured to present a set of individual images and/or individual videos or at least one single video on the image display device to stimulate a number of individual emotions in the subject, and
an emotion observation unit that is configured to evaluate a recording of the face of the subject obtained using an image recording device at least for the purpose of determining when the subject shows an emotional reaction,
wherein the emotion analysis module is configured to ascertain at least a respective reaction time between the stimulation of the respective emotion and the occurrence of the emotional reaction, the reaction times forming the characteristic values of the second biomarker.

28. The diagnostic tool according to claim 26, wherein the further module is a line-of-vision analysis module for evaluating the line of vision of the subject as the second biomarker, wherein the line-of-vision analysis module comprises:
a line-of-vision director that is configured to present at least one image or video on the image display device to direct the line of vision of the subject, and
a line-of-vision observation unit configured to use a recording of the face of the subject obtained using an image recording device to ascertain the line of vision of the subject over time, the line-of-vision response forming the characteristic values of the second biomarker.

29. The diagnostic tool according to claim 26, wherein the at least one further module is an emotion analysis module that evaluates a reaction of the subject to an emotional stimulus as the second biomarker, wherein the emotion analysis module comprises:
an emotion trigger controller configured to present a set of individual images and/or individual videos or at least one single video on the image display device to stimulate a number of individual emotions in the subject, and
an emotion observation unit that is configured to evaluate a recording of the face of the subject obtained using an image recording device at least for the purpose of determining when the subject shows an emotional reaction,
wherein the emotion analysis module is configured to ascertain at least a respective reaction time between the stimulation of the respective emotion and the occurrence of the emotional reaction, the reaction times forming the characteristic values of the second biomarker,
wherein the further module is a line-of-vision analysis module for evaluating the line of vision of the subject as the second biomarker, wherein the line-of-vision analysis module comprises:
a line-of-vision director that is configured to present at least one image or video on the image display device to direct the line of vision of the subject, and
a line-of-vision observation unit configured to use a recording of the face of the subject obtained using an image recording device to ascertain the line of vision of the subject over time, the line-of-vision response forming the characteristic values of the second biomarker,
wherein the emotion analysis module is a first further module and the line-of-vision analysis module is a second further module, and at least the reaction times for the emotional stimuli form characteristic values of the second biomarker and the line of vision over time forms characteristic values of a third biomarker of the subject, the overall result assessment unit being configured to take the characteristic values of the first, second and third biomarkers of the subject as a basis for applying the machine learning algorithm based on artificial intelligence to establish, through comparison with a multidimensional boundary layer, whether the subject has the chronic neurological disorder.

30. The diagnostic tool according to claim 26, wherein the learning algorithm is a support vector machine, a random forest, or a deep convolutional neural network algorithm, the learning algorithm having been trained using a number of first and second comparison datasets comprising characteristic values of the biomarkers, the first comparison datasets being associated with a group of reference persons who have the chronic neurological disorder, and the second comparison datasets being associated with a group of reference persons who do not have the chronic neurological disorder.

31. The diagnostic tool according to claim 26, wherein the diagnostic tool is configured to select and present the set of individual images and/or individual videos or the text for triggering the voice signal, and/or the set of individual images and/or individual videos or the at least one video for stimulating emotion and/or the at least one image or video for directing line-of-vision on the basis of person-specific data on the subject, wherein the voice signal trigger controller is configured to take the age of the subject as a basis for selecting and presenting either the set of individual images and/or individual videos or the text.

32. The diagnostic tool according to claim 26, further comprising a bandpass filter configured to limit the pitch spectrum considered to the range between 30 and 600 Hz.

33. The diagnostic tool according to claim 26, wherein the number of frequency bands is between 6 and 18, preferably is 12.

34. The diagnostic tool according to claim 26, wherein the voice signal analyzer comprises a deep convolutional neural network algorithm, in particular CREPE, or a PRAAT algorithm, to estimate the pitches.

35. The diagnostic tool according to claim 29, wherein the emotion observation unit and/or the line-of-vision observation unit are configured to evaluate the facial recording in real time.

36. The diagnostic tool according to claim 27, wherein the emotion observation unit comprises a facial recognition software based on a compassionate artificial intelligence that is trained for specific emotions.

37. The diagnostic tool according to claim 27, wherein the emotion observation unit is configured to establish, in addition to the reaction time, a reaction type for the respective stimulated emotion, the reaction type being part of the characteristic values of the second biomarker.

38. The diagnostic tool according to claim 27, wherein the emotion trigger controller is configured to stimulate between 4 and 12 emotions, preferably 6 emotions.

39. The diagnostic tool according to claim 28, wherein the line-of-vision director is configured to present the at least one image or video at discrete positions on the image display device in succession or to move said at least one image or video along a continuous path.

40. The diagnostic tool according to claim 28, wherein the line-of-vision observation unit comprises an eye tracking software.

41. A software application for a portable communication terminal, comprising a diagnostic tool according to claim 26, wherein the portable communication terminal is a smartphone or a tablet.

42. A software application on a server, comprising a diagnostic tool according to claim 26, the software application being controllable via a computer network by a browser on an external terminal in order to execute the diagnostic tool.

43. A diagnostic system for use in diagnosing a chronic neurological disorder in a human subject using artificial intelligence, comprising:
a diagnostic tool according to claim 26;
at least one nonvolatile memory containing program code and data that form the diagnostic tool;
a processing unit for executing the program code and processing the data of the diagnostic tool; and
peripheral devices, including:
a voice input device for recording at least one voice signal from the subject for the diagnostic tool;
an image recording device for graphically recording a face of the subject for the diagnostic tool;
an image display device for presenting image data for the subject; and
at least one input means for the subject to make inputs,
the peripheral devices being operatively connected to the processing unit, and the diagnostic tool being configured to at least indirectly control the voice input device, the image recording device and the image display device and to evaluate the recordings from the voice input device and the image recording device.

44. The diagnostic system according to claim 43, wherein the diagnostic system is a portable communication terminal, in particular a smartphone or tablet.

45. The diagnostic system according to claim 43, wherein the processing unit is part of a server that is connected to a computer network and able to be controlled via a browser, and the nonvolatile memory is connected to the server, wherein the peripheral devices are part of an external terminal, in particular a portable communication terminal.

46. The diagnostic system according to claim 45, wherein the external terminal has a further volatile memory, the diagnostic tool being stored partly on the server memory and partly on the terminal memory.

47. A method for operating a software-based diagnostic tool for use in diagnosing a chronic neurological disorder in a human subject using artificial intelligence, comprising the steps of:
providing a superordinate operating software;
using a voice analysis module for ascertaining characteristic values of a first, specifically vocal, biomarker of a voice signal from the subject;
using at least one further module for ascertaining characteristic values of a second biomarker;
connecting an overall result assessment unit downstream of the voice analysis module and the further module;
triggering the voice analysis module and the at least one further module in succession and supplying the ascertained characteristic values thereof to the overall result assessment unit using the operating software;
presenting a set of individual images and/or individual videos or a text on an image display device for the subject by a voice signal trigger controller of the voice analysis module in order to trigger at least one voice signal from the subject in the form of a naming of an object contained in the respective individual image or individual video or in the form of a reading aloud of the text;
recording the voice signal, using a voice recording unit of the voice analysis module, in an audio recording using a voice input device;
evaluating the voice signal in the audio recording, using a voice signal analyzer of the voice analysis module, initially for determining a time at which what pitch occurs, and then ascertaining a prevalence distribution for the pitches over a number of frequency bands of a frequency spectrum considered, the prevalence distribution forming the characteristic values of the first biomarker; and
the overall result assessment unit taking the characteristic values of the biomarkers of the subject as a basis for applying a machine learning algorithm based on artificial intelligence to establish, through comparison with a multidimensional boundary layer, whether the subject has the chronic neurological disorder.

48. The method according to claim 47, wherein the further module is an emotion analysis module for evaluating the reaction of the subject to an emotional stimulus as the second biomarker, wherein the emotion analysis module carries out the following steps:
an emotion trigger controller of the emotion analysis module presents a set of individual images and/or individual videos or at least one single video on the image display device in order to stimulate a number of individual emotions in the subject,
an emotion observation unit of the emotion analysis module evaluates a recording of the face of the subject obtained using an image recording device at least for the purpose of determining when the subject shows an emotional reaction, and
the emotion analysis module ascertains at least the respective reaction time between the stimulation of the respective emotion and the occurrence of the emotional reaction, the reaction times forming the characteristic values of the second biomarker.

49. The method according to claim 47, wherein the further module is a line-of-vision analysis module for evaluating the line of vision of the subject as the second biomarker, and the line-of-vision analysis module carries out the following steps:
a line-of-vision director of the line-of-vision analysis module presents at least one image or video on the image display device to direct the line of vision of the subject, and
a line-of-vision observation unit of the line-of-vision analysis module uses a recording of the face of the subject obtained using an image recording device to ascertain the line of vision of the subject over time, the line-of-vision response forming the characteristic values of the second biomarker.

50. The method according to claim 47, wherein the further module is an emotion analysis module for evaluating the reaction of the subject to an emotional stimulus as the second biomarker, wherein the emotion analysis module carries out the following steps:
an emotion trigger controller of the emotion analysis module presents a set of individual images and/or individual videos or at least one single video on the image display device in order to stimulate a number of individual emotions in the subject,
an emotion observation unit of the emotion analysis module evaluates a recording of the face of the subject obtained using an image recording device at least for the purpose of determining when the subject shows an emotional reaction, and
the emotion analysis module ascertains at least the respective reaction time between the stimulation of the respective emotion and the occurrence of the emotional reaction, the reaction times forming the characteristic values of the second biomarker,
wherein the further module is a line-of-vision analysis module for evaluating the line of vision of the subject as the second biomarker, and the line-of-vision analysis module carries out the following steps:
a line-of-vision director of the line-of-vision analysis module presents at least one image or video on the image display device to direct the line of vision of the subject, and
a line-of-vision observation unit of the line-of-vision analysis module uses a recording of the face of the subject obtained using an image recording device to ascertain the line of vision of the subject over time, the line-of-vision response forming the characteristic values of the second biomarker, and
wherein the emotion analysis module is a first further module and the line-of-vision analysis module is a second further module and these modules are triggered in succession, at least the reaction times for the emotional stimuli forming characteristic values of the second biomarker and the line of vision over time forming characteristic values of a third biomarker of the subject, and the overall result assessment unit taking the characteristic values of the first, second and third biomarkers of the subject as a basis for applying the machine learning algorithm based on artificial intelligence to establish, through comparison with a multidimensional boundary layer, whether the subject has the chronic neurological disorder.
